(19) United States
(12) Patent Application Publication
(10) Pub. No.: US 2008/0044919 A1
(43) Pub. Date: Feb. 21, 2008

(54) Analyte Detection Using Time-Resolved Photon Counting Fluorescence

(76) Inventors: Robert M. Dowben, Providence, RI (US); Martin L. Alford, Kaufman, TX (US)

Correspondence Address: Richard D Zimmerman;Chance Ruttenberg & Freedman, One Park Row Suite 300, Providence, RI, 02903, US

(21) Appl. No.: 11/660,115

(22) PCT Filed: Aug. 12, 2005
(86) PCT No.: PCT/US05/28820

§ 371 Date: Feb. 12, 2007

Related U.S. Application Data
(60) Provisional application No. 60/601,576, filed on Aug. 13, 2004.

(51) Int. Cl. G01N 33/48; G01N 21/17; G01N 21/64

(52) U.S. Cl. 436/94; 356/433; 436/172

(57) ABSTRACT

Samples are exposed to a fluorescent probe that binds to a known target analyte. Unbound probe is removed and the sample is exposed to a pulsed laser light of preferred wavelength, where the laser light is split into background and sample beams, which are separately measured by two photomultiplier tubes connected to a gated scanning dual channel photon counter Measurements are made by time-resolved photon counting, where individual photons are detected and counted as an electronically synchronized function of the time after each light pulse, and data are summated over a number of light pulses, to detect and quantify the target analyte.

### CROSS-REFERENCE

The present application claims priority based on U.S. Provisional Patent Application Number U.S. 60/601,576 filed Aug. 13, 2004, titled Analyte Detection Using Time-Resolved Photon Counting Fluorescence by inventor Marlin L. Alford and Robert M. Dowben, which application is incorporated herein in its entirety by this reference.

### BACKGROUND

Advances in biology, medicine and other sciences have rendered the detection of specific nucleic acid sequences, including DNA and RNA, of great importance. Detection of specific known nucleic acid sequences permits identification of viruses, bacteria and other infectious agents, as well as genetic defects. Identifying the presence of specific nucleic acid sequences also is important to reveal the presence of particular genes having biological significance.

DNA testing is a comprehensive term that includes a number of testing formats. A very large group of DNA testing procedures involves the determination of the nucleotide base sequences. Another different group of DNA tests involves the determination of whether a sample contains any amount of a specific, known DNA sequence, and often quantifying the amount of the specific DNA in the sample. Now that the sequence of the entire human genome has been determined, many important sequences identifying infectious agents are known, the sequences of many genetic modifiers are known, and an increasing number of other sequences are becoming known, there will be more and more situations where it is important to rapidly and simply identify the presence or absence of a specific gene sequence and to easily roughly quantify it in various samples. The present technology involves the latter type of DNA testing. It detects whether or not the DNA or RNA in an unknown sample hybridizes with a known, standard complementary DNA (c-DNA) sequence. There are other uses of this detection method, for example, sequences of viruses, bacteria and other infectious agents are known and testing of this type establishes whether a patient is suffering from an infection by a specific virus or bacteria, and often the strain of the infectious agent. It will be appreciated that the system described here can be used for many other kinds of testing besides DNA and RNA. The present invention allows assays that are simple, very sensitive and can be performed very rapidly and adapt themselves to screening.

At the present time, the major limitations of DNA and RNA testing methods used for screening are the complexity of the assay, the time required to perform the assay, its sensitivity, and the cost of the procedure. The amount of DNA available for testing is extraordinarily small, much less than the threshold sensitivity of generally available measurement systems. The strategies presently used to get around the limited sensitivity of the measurement systems is to first amplify the amount of nucleotide in the sample, that is to first create a larger sample of DNA by synthesizing more copies using PCR (polymerase chain reaction) or other amplification procedures such as branched chain reaction, or linear chain amplification. In spite of the recent availability of compact automated equipment, which is often expensive, all of the amplification methods require complex off line processing, and they require considerable technician time and effort. The procedures must be carried out under rigorous conditions to prevent contamination, for example, from nucleotides in other samples, from DNA in droplets in the air or from surface contamination. PCR is highly susceptible to contamination and suffers from variations in amplification efficiency not fully understood at this time. [A. K. et al., Brit. Rev. Biochem. Biophys. 26:301-334 (1991); Peccoud, J. et al., Biopys. J. 18:973-978 (1990); Taranger, J. et al., Pediatr. Infect. Dis. 13:936-937 (1994); Wilke, W. W. et al., Clin. Chem. 41:622-623 (1995)] Various control runs are required for reliable results. After amplification, the DNA product still requires a number of steps for hybridization and then quantitative measurement whether by use of radioactive isotopes, fluorescence, luminescence, etc. The present invention discloses a means of very sensitive measurement of DNA or RNA, so sensitive that the DNA or RNA content of most samples can be measured directly without the need of prior amplification.

Other known amplification technologies include the branched DNA (bDNA) assay, in which the signal generated by labeled probes is amplified through the use of nucleic acid multimers. However, when several commercially available assays for the detection of hepatitis B virus (HBV) DNA, including a bDNA assay, were compared, all of the assays tested had poor accuracy and/or sensitivity. [See Zaaijer et al., J. Clin. Microbiol. 32:2088-2091 (1994)]

Other assays have been developed more recently which use fluorescent-labeled probes and photon counting for the detection of specific nucleic acids. Although these assays claim to achieve greater accuracy and sensitivity over conventional methods, the majority of these assays have not been tested on a mixed population of nucleic acids. [Perkins, et al., Science 1994, 264, 822-826; Larson, et al., Phys. Rev. E1997, 55, 1794-1797; Castro, et al., Anal. Chem. 1993, 65, 849-852; Goodwin, et al., Nucleic Acids Res. 1993, 21, 803-806; Castro, et al., Anal. Chem. 1995, 67, 3181-3186; Haab, et al., Anal. Chem. 1995, 67, 3253-3260], Oehlenschlager, et al., Proc. Natl. Acad. Sci. USA, 93, 12811-12816]. Those assays that have been tested on a mixed population of nucleic acids typically require the use of several probes or other complicated means for nucleic acid detection. For example, Castro (Anal. Chem. 1997, 69, 3915-3920) discloses single molecule detection of specific nucleic acids in unamplified genomic DNA using photon counting. The method consists of using two nucleic acid probes complementary to different sites on a target DNA sequence. Castro's method requires that two fluorescent labeled probes be hybridized in close proximity to one another on a single DNA molecule before detection of label is counted as positive for a specific DNA. Thus, coincident detection of both dyes provided the necessary specificity to detect an unamplified, single-copy target DNA molecule in a homogeneous assay. However, Castro's system requires complex, time consuming sample preparation.

Known methods of detecting the presence and quantification of nucleic acid sequences lack sensitivity and simplicity, and are time-consuming, costly and error-prone. What is needed is a faster, simpler, more sensitive, more accurate economical device and method to detect specific known nucleic acid sequences without the need for amplification.

### SUMMARY

The present invention is directed to a sensitive assay for detecting the presence of small amounts of a specific known nucleic acid sequence or sequences in a mixture of unamplified sample nucleic acids. The present assay is more efficient than conventional nucleic acid assays because the assay does not include any further separation of the mixture of sample nucleic acids once the mixture has been isolated from its original source, and because the assay does not include any nucleic acid amplification step. In addition, the present assay is more sensitive than conventional nucleic acid assays in detecting small amounts of specific known nucleic acids because the assay uses fluorescent-labeled nucleic acid probes, and photon counting to indirectly detect specific nucleic acid sequences. In particular, one aspect of the present assay is directed to an assay for detecting a specific nucleic acid using photon counting.

The technology is a time-resolved photon counting fluorescence measuring system that can quantify various analytes. It has been used specifically for the analysis of hepatitis-C, an enveloped RNA virus, and experiments were done for detecting some other nucleotide sequences. Known DNA measuring systems are limited by lack of sensitivity, lack of simplicity and cost of the assay procedure. A simpler, more sensitive, economical measuring system represents a major improvement in technology. There are, of course, other potential uses for this measuring system.

One effort using the invention was to assay a known viral RNA or DNA; hepatitis-C was selected for a proof of principle. The assay is based on hybridizing the sample RNA with a synthesized complementary DNA (c-DNA) containing a specific base sequence defining the DNA/RNA of interest, that has been labeled with the long-lived fluorescent dye. If the structure of the unknown sample corresponds in a complimentary way to the structure of the labeled c-DNA, a double stranded complex will be formed that can be quantified by time-resolved fluorescence as described above. There are several ways in which the c-DNA can be fluorescently labeled. A series of amines can be attached at the 5′ end of the c-DNA, for example, a short stretch of polylysine containing about 20 free epsilon amino groups. The fluorescent dye can then be covalently attached to the free amino groups on the lysines. This has the advantage of labeling a single DNA molecule by several dye molecules, and also the advantage that the dye molecules are not in the region of the interface between the two nucleotide strands and do not interfere with hybridization and also that the nucleotides do not quench the fluorescence.

A RNA or DNA test begins with isolating the nucleotide in the sample using commercially available extraction kits, and then unwinding it under denaturing conditions to make the nucleotide single stranded. If the nucleotide in the sample is small (viruses contain only 4 to 12 thousand base pairs) it can be used directly; if the nucleotide is large, it must first be cleaved into smaller fragments by use of restriction enzymes. The restriction enzymes used are selected to produce the optimum fragment for the sequence of interest. The nucleotide is then captured on a solid matrix, such as a charged nylon bead or a nylon membrane. The fact that the samples are fixed in space is a decided advantage in fluorescent counting of a small number of molecules. When the sample is fixed on the solid matrix, the matrix is blocked to prevent any further DNA binding. After blocking, the nylon membrane or bead is placed in a reaction vial and a premeasured amount of fluorescent labeled c-DNA in hybridization buffer is added and the sample incubated. The hybridization process is completed in 5 minutes or less. If the gene of interest is present in the sample DNA or RNA, the c-DNA will hybridize and form a double stranded nucleotide.

There are several ways in which the c-DNA can be fluorescently labeled. The unique fluorescent dyes used are discussed below. After incubation, the matrix is washed, removing all unbound c-DNA and non-specifically bound c-DNA. The nylon membrane or bead is suspended in a polar nonaqueous solvent such as dimethylformamide. The fluorescence of the membrane matrix with hybridized DNA is then quantified in the novel, very sensitive, time-resolved photon counting fluorescence system. If the sample did not contain the gene of interest, virtually no counts will be recorded, indicating a negative result. If the gene is present however, the counts will be very high, indicating a positive result. The total process from isolation of the DNA or RNA, blocking the matrix, to obtaining a result can be accomplished less than one hour, and the assays can be run in batches.

The new photon detection system described below achieves routine sensitivities using 100 microliter sample volume of better than 10 E-17 molar, and with the system tuned up, the sensitivities were better than 10 E-18 molar. The system was used for identifying hepatitis-c virus directly in whole blood. In a large batch of whole blood samples from Baylor Hospital in Dallas, Tex., all positive samples were identified, and there were no false positives.

The device and method use a time-resolved photon counting system that has continuous real-time background subtraction, in which the photo multiplier tube is selected for rapid electron cathode to first dynode transit time. The device also uses a cathode anode voltage divider providing a somewhat higher voltage to the first dynode. The photomultiplier tube (“PMT”) may be thermo-electrically cooled (such units are manufactured by Hamamatsu Corp.). Improved results are achieved by labeling of nucleic acid by covalent reaction of the fluorescent probe to the epsilon amino groups of a polylysine oligomer attached to the 5′-end of the DNA, and by labeling it with multiple fluorescent probe molecules. This device and method allows precise, accurate, rapid, sensitive, and economical detection of specific known nucleic acid sequences.

The advantages offered by the present assay make it especially suited for use in the clinical setting, where rapidity, accuracy and sensitivity commonly are crucial. A further advantage of the present assay is that the assay may be performed in microtiter plates and several of its steps may be automated, thereby saving costs in labor and reagents. It is contemplated that the present assay may be used to detect target nucleic acids that are indicative of a wide variety of pathogens, including but not limited to viruses, prions, bacteria, protozoans, helminths and the like. The target nucleic acids may be detected in samples obtained from human or animal blood, sputum, urine, feces, spinal fluid, etc. Of particular interest are target nucleic acids indicative of human immunodeficiency virus (HIV), hepatitis A virus (HVA), hepatitis B virus (HBV), hepatitis C virus (HCV), herpes simplex virus (HSV), cytomegalovirus (CMV), human papilloma virus (HPV), human herpes virus (HHV), Chlamydia trachomatis, Neiseria meningiditis, Neiseria gonorrhea, Mycobacterium tuberculosis, and Plasinodium. Of particular interest is the potential use of this technology for detecting agents used in biological warfare or terrorism. A wide variety of other pathogens and target nucleic acids for each can readily be selected by a person skilled in the art for a given application.

It is further contemplated that the present assay be used to determine the number of infectious organisms present in a particular volume of patient tissue or fluid, where such a determination is useful in choosing a proper course of patient treatment. For example, the present assay may be used to determine the viral load in a patient infected with HIV or HCV. Similarly, the present assay may be used to detect genetic sequences associated with antibiotic and/or drug resistance in order to better modify the treatment of patients infected with various microorganisms or undergoing certain chemotherapies. The present assay is also especially suited for a variety of non-clinical uses. For example, the present assay can be used to detect bacterial DNAs in recombinant pharmaceuticals (such as insulin, bovine growth hormone), recombinant vaccines (such as hepatitis A vaccine) and other recombinantly prepared products for which the FDA and WHO recommend that the final product contain less than 100 pg host cell DNA per dose. Other non-clinical uses include testing for the presence of pathogens, such as Salmonella and Escherichia coli, in water and food supplies. The present assay is further suited for testing for the presence of genetically-engineered or modified plants or animals. Such testing would be useful for monitoring the presence or propagation of recombinant genes into the environment. The present assay will further find use in forensic screening and other forensic testing. Other uses for the present assay will be recognized by those skilled in the art.

### DETAILED DESCRIPTION OF THE DRAWING

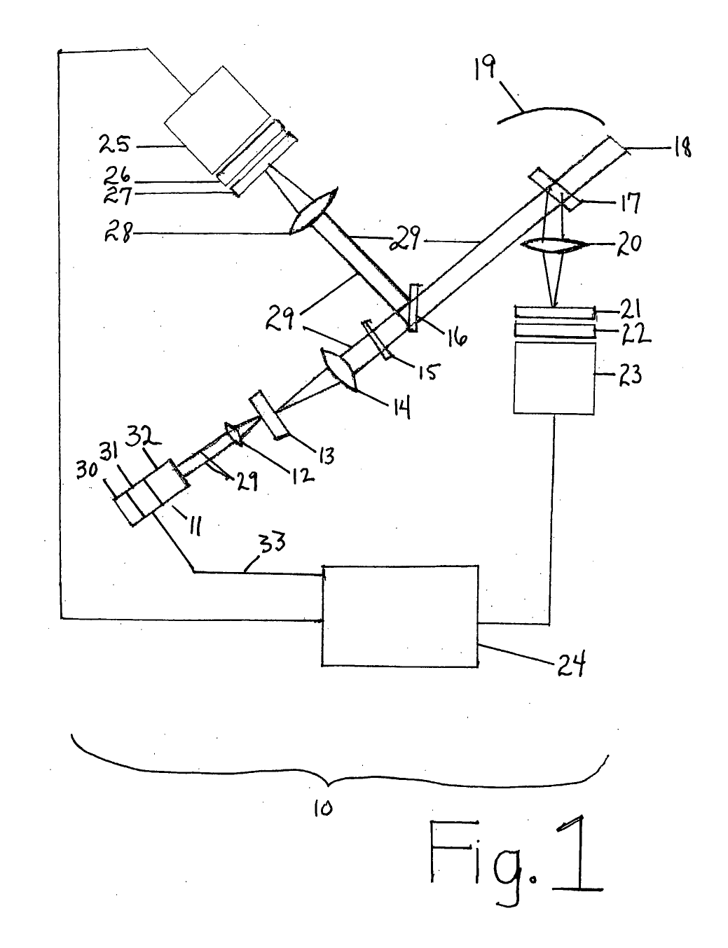

FIG. 1 is a schematic view of the analyte detection system.

### DESCRIPTION

The practice of the present assay can employ, unless otherwise indicated, conventional methods of molecular biology and recombinant DNA techniques within the skill of the art. Such techniques are explained fully in the literature. See, e.g., Sambrook, et al., Molecular Cloning: A Laboratory Manual (2nd Edition, 1989); DNA Cloning: A Practical Approach Vols. I & II (D. Glover, ed.); Oligonucleotide Synthesis (Gait, ed., 1984); Nucleic Acid Hybridization (B. Hames & S. Higgins, eds., 1985); Transcription and Translation (B. Hames & S. Higgins, eds., 1984); Perbal, A Practical Guide to Molecular Cloning (1984); Fundamental Virology, 2nd Edition, Vols. I & II (B. N. Fields and D. M. Knipe, eds.); the series, Methods In Enzymology (Academic Press, Inc.); Methods in Enzymology (1987) 154 and 155 (Wu and Grossman, and Wu, eds., respectively); Mayer & Walker, eds. (1987), Immunochemical Methods In Cell And Molecular Biology (Academic Press, London); and Handbook Of Experimental Immunology Vols. I (Weir and Blackwell, eds., 1986).

As used herein, a “biological sample” refers to a sample of tissue or fluid isolated from an individual or animal, including but not limited to, blood, plasma, serum, fecal matter, urine, bone marrow, bile, spinal fluid, lymph fluid, samples of the skin, external secretions of the skin, respiratory, intestinal, and genitourinary tracts, tears, saliva, milk, blood cells, organs, biopsies, and also samples of in vitro cell culture constituents including, for example, conditioned media resulting from the growth of cells and tissues in culture medium, e.g., recombinant cells, and cell components. Biological samples contemplated for use in the present assay also include biological fluids or solids isolated from plants, food stuffs and environmental materials, such as soil samples or water supplies.

As used herein, “sample nucleic acid” refers to a nucleic acid isolated from a biological sample. Nucleic acids comprising sample nucleic acids include DNAs, such as genomic DNA (gDNA) and mitochondrial DNA (mtDNA); and RNAs, such as messenger RNA (mRNA). Sample nucleic acids used in the present assay may be isolated and prepared for hybridization by a variety of molecular biology techniques known to those skilled in the art, including but not limited to proteinase K/SDS, chaotropic salts, etc. Thus, the sample nucleic acid is provided in single-stranded form for analysis. Where the sequence is naturally present in single-stranded form, denaturation will not be required. However, where the sequence is present in double-stranded form, the sequence will be denatured. Denaturation can be carried out by various techniques known to those skilled in the art, such as acids, alkali (generally from about 0.05 to 0.2M hydroxide), formamide, salts, heat, or combinations thereof. It may be advantageous to reverse transcribe mRNAs into complementary DNAs (cDNAs) to prevent degradation of sample RNA due to environmental factors, such as the presence of RNases, etc. Methods for reverse transcription of mRNAs are known in the art. It may also be of advantage to decrease the average size of the sample nucleic acids by enzymatic, physical or chemical means, e.g., with restriction enzymes, sonication, chemical degradation (for example, with metal ions), etc. The fragments may be as small as 0.1 kb, usually being at least about 0.5 kb and may be 1 kb or higher.

As used herein, “solid matrix” refers to a solid substrate such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, and the like. When particles are used, they can be of a size in the range of about 0.4 to about 200 microns, or in the range of about 0.8 to about 4.0 microns. The particles can be made from any convenient material, such as latex or glass. Microtiter plates and nitrocellulose or nylon membranes are preferred solid matrices. The sample nucleic acids can be stably attached to the solid matrix through functional groups by known procedures. For example, the nucleic acid can be bound directly to the solid matrix, such as in a “dot blot” or a “slot blot.” Alternatively, the solid matrix can be first reacted with a solid phase component (e.g., one or more common chemicals) to facilitate binding of the sample nucleic acids to the solid matrix. Such molecules and methods of coupling these components to a solid matrix are well known to those of ordinary skill in the art.

As used herein, “denaturing conditions” refers to those conditions known to skilled artisans which produce the denaturation of a double-stranded nucleic acid and/or prevent secondary structure in or rehybridization of a single-stranded nucleic acid. Denaturing conditions can include the use of acids, alkali (generally from about 0.05 to 0.2M hydroxide), formamide, salts, heat, or combinations thereof to effect denaturation.

As used herein, “nucleic acid probe” refers to a single-stranded nucleic acid that is complementary to (specific for) at least a portion of a target nucleic acid sequence. Nucleic acid probes can be oligonucleotide sequences, intermediate and full length single-stranded DNA sequences. Nucleic acid probes can be prepared by chemical synthesis or from natural or recombinant sources, such as cDNA libraries, using techniques known in the art. It will be appreciated that the binding sequences need not have perfect complementarity to provide homoduplexes.

Nucleic acid probes intended for use in the present assay to detect specific target nucleic acid sequences include, for example, the pHE63 probe for HBV detection (as disclosed in U.S. Pat. No. 5,614,362); nucleic acid probes specific for all or a portion of the gG1 and gG2 genes for HSV detection (as disclosed in McGeoch et al., J. Mol. Biol. 181:1-13 (1985); nucleic acid probes specific for all or a portion of the VP16 gene for HSV-1 and HSV-2 detection (as disclosed in Campbell et al., J. Mol. Biol. 180:1 (1984); the GCP probes for N. gonorrhea detection (as disclosed in U.S. Pat. No. 5,614,362); the TEM-1 and TEM-1 NH probes for tetracycline resistance in a variety of bacteria (as disclosed in U.S. Pat. No. 5,614,362). In addition, a wide variety of nucleic acid probes which may be used in the present assay are currently available from commercial sources, including Molecular Probes, Eugene, Oreg.

As used herein, “label” refers to a fluorescent molecule which is capable of exhibiting fluorescence in a detectable range, such that the fluorescence emission will always be red-shifted in the spectrum with regard to the excitation wavelength. It is desirable that the fluorescent labels used in the present assay have: high absorbance; high fluorescence quantum yield; resistance to photobleaching; minimal change of fluorescent properties with changes in the polarity of solvent or local environment; longer fluorescent lifetime. Suitable dyes for use as labels in the present method are modified to contain a chemically reactive group so that they can be covalently linked to substrates of interest.

The term “nucleic acid” refers to all nucleic acids, and includes sample nucleic acids.

The term “probe” refers to material that will bind to a specific known target analyte or a target analyte binding material, and to a label. The term probe includes nucleic acid probes.

The term “fluorescent-labeled probe” refers to a label bound to a probe.

The term “target analyte binding material” refers to a material that will bind to a target analyte and to a fluorescent-labeled probe, and includes antibodies and antibody fragments.

As used herein, “stringent conditions” refers to conditions in which the nucleic acid probe will not form duplexes with nucleic acid sequences wherein a fraction of the bases are non-complementary. The stringency of the wash medium can be controlled by temperature, salt concentration, the solvent system, etc. [See Sambrook, et al., Molecular Cloning: A Laboratory Manual (2nd Edition, 1989).] Thus, depending upon the length and nature of the sequence of interest, the stringency can be varied according to known practices.

“Pulsed” is used herein to mean a repetitive finite duration of time under which the sample is exposed to the light source. The pulse duration is provided as the full duration at half maximum of the total light intensity and is 1 nanosecond, alternatively about 0.5 nanoseconds, or alternatively about 0.1 nanoseconds. “Repetition” is used herein to mean the frequency of pulses to which the sample is exposed, and is about 10 megaHertz, alternatively about 1 megaHertz, alternatively about 0.5 megaHertz, alternatively about 0.1 megaHertz. In another embodiment, a solid-state diode laser is used as the light source. The output is light with wavelength about 700 nm, alternatively about 455 nm, and alternatively about 360 nm.

All publications, patents and patent applications cited herein, whether supra or infra, are hereby incorporated by reference in their entirety.

The analyte detection system (10) as shown in FIG. 1, comprises a laser light source (11) that has an associated power supply (30), a pulse generator (31) and laser (32) for producing exciting light pulses; a photon detection unit as described below, and a photon counter (24), such as a Stanford Research Systems SR400Dual Channel Gated Photon Counter, or similar counter.

The SR400 Dual Channel Gated Photon Counter is a microprocessor controlled instrument comprising two independent channels of pre-amplifiers and amplifiers, pulse height discriminators for each channel, gated scanning for time-resolved measurements, and two independent counting channels.

The laser light source (11) selected depends on the fluorescent probe used in a particular assay. For example, for use of 1-hydroxypyrene-3,6,8-trisulfonyl chloride sulfonamides (Ex 455, Em 511) mentioned below, a 455 nm blue diode laser available from several suppliers can be used. Europium labeled analytes, 1-pyrenehexanoic acid labeled analytes, and pyrenesulfonic acid labeled analytes have excitation maxima at about 360 nm and can be excited by pulsed 350 nm lasers, or by 700 nm pulsed diode lasers using frequency doubling crystals also available from many suppliers. The lasers can be pulsed over a wide range of frequencies. Pulsing at 0.1-2 kiloHertz worked well. A line (33) from the laser pulse generator (31) to the photon counter (24) serves to gate the photon counter.

Photomultiplier tubes (PMT) (23, 25) are very sensitive devices that can detect single photons (Photon Counting Using Photomultiplier Tubes, Technical Publication, Hamamatsu Corp., April 2001). The sensitivity of the measurements depends greatly on having a very low level of nonspecific background counts (noise) most of which arises in the PMT itself. Background counts are increased during the period immediately following exposure to light and the PMT should be dark adapted. But even after storage in the dark for several hours, there are some spontaneous discharges of noise. The present technology teaches methods for minimizing the level of background counts, particularly for measurements made when the PMT is not completely dark adapted.

At low light levels, individual photons produce discrete electrical pulses that can be amplified and counted. Several strategies are used to minimize noise in PMT systems. Counts from fluorescence photons (real data counts) tend to have a small range of similar amplitudes after amplification, while counts from background noise have a wide range of amplitudes. Background noise can be partially rejected by using pulse height discriminators after amplification and before counting to reject counts with amplitudes out of range for fluorescence photons. Amplitude discrimination of the photon pulses is one strategy used to reduce background.

Photon Detection Unit:

The detailed arrangement of the photon detection unit is shown in the accompanying FIG. 1. Light from the laser (11) is directed down an optical path after being focused by two lenses (12, 14). The light paths are contained in enclosures (29) to reduce extraneous light. A light chopper or shutter (13) can be used to block the laser light. A neutral grey wedge (15) is used to adjust the light intensity. A beam splitter (16) breaks the light into two beams, one directed to the data collecting PMT (23), and the other directed to the background measuring PMT (25). Dielectric band pass filters (21,27) (Corion Optics) centered on the fluorescence probe maximum emission wavelength are positioned in front of both PMT's, as are long pass filters (22,26) (Corion Optics). The sample is contained in or on a known container (17) or support, such as a measuring cuvette, test tube or slide. A flat plain mirror (18) at an end of the optical pathway reflects light back toward the container (17) to minimize light losses. Fluorescent light from the container (17) is focused on the data collecting PMT (23) by a lense (20). A parabolic reflecting mirror (19) is focused on the data collecting PMT (23), and is a decided advantage in marshalling all of the fluorescent light emitted into the measuring PMT. Components for which no source is given can be obtained from many suppliers such as Edmund Scientific.

Band pass (21, 27) and low pass (22, 26) filters largely block light from the exciting laser light pulses but do not completely do so. Dielectric filters improve the performance. The sensitivity of the measurements depends greatly on having a very low level of nonspecific background counts (noise). As noted above, the PMT must be shielded from light, and dark adapted, to minimize noise and achieve high sensitivity. However, in the present application, frequent intense light pulses must be used in order to obtain measurements in a reasonable time, which markedly raises the background counts and limits the sensitivity. The photomultipliers actually dark adapt during the 150 to 250 nsec measurement time, i.e., the background noise level is high immediately after the light pulse and gradually decreases during the measurement period. The present invention teaches several strategies to minimize the effect of the variable noise following the light pulse.

In addition to using pulse height discrimination to reject noise, the PMT unit of the present invention copes with the problem of variable background noise during the course of the response to a single light pulse. Because the photomultipliers are dark adapting during the course of the measurements, the second strategy for minimizing noise is to use dynamic, real-time background subtraction. Two identical side window bialkali cathode photomultiplier tubes with short cathode anode distances, and short electron transit time were normalized as regards sensitivity to light intensity by adjusting the anode voltages. As noted above, a beam splitter (16) is used in illuminating each PMT. In practice, the background measuring PMT (25) was illuminated only with the exciting laser light pulses and generates a true overall background count. The sample container (17) is placed in the pathway of the data collecting PMT (23) which thus registers true fluorescence emission photon counts plus background. The background counts of the first PMT (25) are subtracted from the measured counts of the active data collecting PMT (23) continuously. Those experienced in the art will appreciate that other methods can be used to obtain real-time background subtraction. For example, a photoelastic modulator (Hinds Instruments) could be used that polarizes the light in one direction for background and in the other direction for data/sample so that one PMT provides continuous dynamic background subtraction.

Small side-window photomultiplier tubes such as the R6358 (Hamamatsu Corp.) are preferred because they dark adapt most rapidly. Individual PMT's are selected for very low background noise. Noise can be still further minimized by sweeping the background noise electrons emitted by the photocathode up as quickly as possible. Therefore, instead of using a voltage divider to the dynodes with equal voltage increments, a slightly higher voltage is applied to the first dynode to help gather up the background electrons more quickly. Thermo-electrically cooling the photomultiplier tube to −0° C. or less will still further reduce background (cooling units are available from Hamamatsu Corp.).

One embodiment used a small GaAs cathode photomultiplier tube (H7421-40 Hamamatsu Corp.) with a high work function, high quantum counting efficiency, and other salutary features for detection of emitted photons.

Fluorescent Probes

Many substances exhibit fluorescence which occurs when an outer electron of the material is excited by a light photon, and subsequently the electron decays back to the ground state emitting a photon of light. The emitted light is shifted to the red compared to the exciting light. The light emitting photon decay is a first-order reaction. Most fluorescent materials have fluorescence life-times of one nanosecond (nsec) or less; after a brief light pulse, almost all fluorescence decays within 4 nsec. There are some unusual materials with longer fluorescence life-times. Key issues in the present technology are the fluorescent dye used, the method of labeling, and the novel, very sensitive time-resolved photon counting fluorescence system described in more detail below used for measurement.

The fluorescent dyes used for analyte quantification have long fluorescent life times greater than 10 nanoseconds (nsc), are resistant to photobleaching, and have large Stokes shifts greater than 15 nm. Measurements are made by time-resolved photon counting. In the present technology, the fluorescently labeled analytes are excited by lasers emitting a series of very short (<1.0 nsec fwhm) intense light pulses at high repetition rates, of the order of several hundred Hertz. Individual photons are detected by sensitive photomultiplier tubes (PMT), and counted as an electronically synchronized function of the time after each light pulse, and the data are summated over a number of light pulses. Electronic circuits synchronize the laser pulses and intervals of photon counting. In practice, the detection system is turned off and no photons are counted for the first 5-12 nsec after the exciting light pulse. During this “dead time” light from the light source and virtually all of the interfering background fluorescence decays. The photons that are counted after 5-12 nsec are virtually entirely from the long-lived fluorescent dye labeled analytes, thus interfering fluorescence is almost entirely eliminated from the measurement (O'Connor, D. V. and Phillips, D., Time-correlated Single Photon Counting, Academic Press, 1986).

One group of fluorescent materials with long fluorescent life-times are the rare earths, particularly europium and terbium. Excitation of the rare earths involves the movement of an internal electron from the D shell to the F shell. Because an internal electron is involved, excitation is not by light, but by fluorescence resonance energy transfer from another adjacent fluorescent material that emits at or to the blue of the rare earth excitation maximum wavelength. In practice, the rare earth is used bound to a chelating agent that serves to (i) solubilize the rare earth by chelation, (ii) provide the resonance energy to excite the rare earth molecule, and (iii) provide a chemically reactive group that will form a covalent chemical bond to the analyte to be measured.

Several suitable chelating agents are commercially available such as diethylene-triamine-pentacetic acid isothiocyanate. These chelating agents were developed principally for use with the rare earth gadolinium for contrast enhancement of MRI'S. When the electron of the rare earth molecule is excited, it changes its direction of rotational spin forming a “triplet”. The excited photon is “forbidden” to decay back to the ground state until its spin reverses back to the original direction, usually as a result of a quenching collision. As a result, the rare earth molecules have very long fluorescent life-times, as long as more than 1,000 nsec.

The lanthanides have been used for DNA labeling and quantification (reviewed by Selvin, P. R., Lanthanide-labeled DNA, Topics in Fluorescence Spectroscopy, 2003, 7:177-212). The use of lanthanides is limited by the long fluorescent lifetimes that make long exposure times necessary, the instability of the lanthanide chelates, and the bulkiness of the lanthanide chelates that makes labeling more difficult.

A sometimes more useful group of chemical compounds with long fluorescent life-times for the technology disclosed here are certain pyrene compounds. The index compound of one group of pyrenes is 1-pyrenehexanoic acid (available from Fluka Chemie, Buchs, Switzerland). Molecular orbital analysis shows that pyrene itself is a flat molecule with C2h point group symmetry. The addition of the hexanoic functional group lowers the symmetry to C1h or even no symmetry. In practice this means that excitations lead to a charge transfer complex in which the stability of the molecule in the excited state is enhanced, particularly in polar solvents. Thus, 1-pyrene-hexanoic acid and related compounds have fluorescent life-times in the range of 160-180 nsec. 1-Pyrenehexanoic acid can be converted to the succinimidyl ester that in turn will form stable chemical covalent bonds with amino groups on substances to be analyzed. Pyrenesulfonic acid (also available from Fluka) is the index compound of another group of useful pyrene compounds. Pyrenesulfonic acid can be converted to the sulfonyl chloride that in turn will form chemically stable sulfonamides with amino groups on analytes. Such sulfonamides have fluorescent life-times in the range of 26-60 nsec. The pyrene dyes are resistant to photobleaching and have large Stoke's shifts.

In the present technology, the fluorescently labeled nucleotide sequences are excited by lasers emitting a series of very short (<1.0 nsec fwhm) intense light pulses at high repetition rates, of the order of several hundred Hertz. Europium labeled analytes, 1-pyrenehexanoic acid labeled analytes, and pyrenesulfonic acid labeled analytes have excitation maxima at about 360 nm and can be excited by pulsed 360-370 nm lasers, or by 720 nm pulsed diode lasers using frequency doubling crystals. We have also labeled nucleotide sequences with 1-hydroxy oxypyrene-3,6-disulfonic acid-8-sulfonyl-beta-alanine succinimidyl ester that has a excitation maximum at 455 nm and can be excited by pulsed blue diode lasers.

Time-Resolved Photon Counting:

Measurements are made by time-resolved photon counting, i.e., individual photons are detected and counted as an electronically synchronized function of the time after each exciting laser light pulse, and the data are summated over a number of light pulses. In practice, the detection system is turned off and no photons are counted for the first 8-12 nsec after the exciting light pulse. During this “dead time” virtually all of the fluorescence from sources other than the probe decays. The photons are counted after 12 nsec and are virtually entirely due to the long-lived fluorescent dye labeled analytes, thus almost entirely eliminating interfering fluorescence from the measurement.

Two embodiments of the technology, one using the side window R 6358 PMT, and one using the GaAs PMT, have been built with routine sensitivities using 100 microliter sample volume of better than 10 E-17 molar, and with the system tuned up, the sensitivities were better than 10 E-18 molar. The detailed arrangement of the measuring system is shown in the accompanying FIG. 1 and explained above. The present technology teaches methods for minimizing the level of background counts that can be reduced to less than 20 counts/second.

One effort using the invention was to assay a known viral RNA or DNA; hepatitis-C was selected for a proof of principle. The assay is based on hybridizing the sample RNA with a synthesized complementary DNA (c-DNA) containing a specific base sequence defining the DNA/RNA of interest, that has been labeled with the long-lived fluorescent dye. If the structure of the unknown sample corresponds to the structure of the labeled c-DNA, a double stranded complex will be formed that can be quantified by time-resolved fluorescence as described above. There are several ways in which the c-DNA can be fluorescently labeled. A series of amines can be attached at the 5′ end of the c-DNA, for example, a short stretch of polylysine containing about twenty free amino groups. The fluorescent dye can then be covalently attached to the free amino groups on the lysines.

A RNA or DNA test begins with isolating the nucleotide in the sample using commercially available extraction kits, and then unwinding it under denaturing conditions to make the nucleotide single stranded. If the nucleotide in the sample is small (viruses contain only 4 to 12 thousand base pairs) it can be used directly; if the nucleotide is large, it must first be cleaved into smaller fragments by use of restriction enzymes. The restriction enzymes used are selected to produce the optimum fragment for the sequence of interest. The nucleotide is then captured on a solid matrix, such as a charged nylon bead or a nylon membrane. The fact that the samples are fixed in space is a decided advantage in fluorescent counting of a small number of molecules. When the sample is fixed on the solid matrix, the matrix is blocked to prevent any further DNA binding. After blocking, the nylon membrane or bead is placed in a reaction vial and a premeasured amount of fluorescent labeled c-DNA in hybridization buffer is added and the sample incubated. The hybridization process is completed in 5 minutes or less. If the gene of interest is present in the sample DNA or RNA, the c-DNA will hybridize and form a double stranded nucleotide.

After incubation, the matrix is washed, removing all unbound c-DNA and non-specifically bound c-DNA. Only double stranded hybridized DNA remains attached to the matrix. The last step of the procedure is to determine the fluorescence of the matrix with hybridized DNA attached. The nylon membrane or bead is suspended in a polar nonaqueous solvent such as dimethylformamide and the fluorescence measured. Alternatively, the matrix with the double stranded DNA can be dried and the fluorescence determined by reflectance. Still further, the double stranded DNA can be removed from the matrix and solubilized with 0.1 M NaOH, the solution transferred to a cuvette and the fluorescence determined. Those knowledgeable in the art will appreciate that there are many ways in which the fluorescence of the double stranded DNA can be determined. If the sample did not contain the gene of interest, virtually no counts will be recorded, indicating a negative result. If the gene is present however, the counts will be very high, indicating a positive result. The total process from blocking the matrix to obtaining a result can be accomplished in less than two hours, and the assays can be run in batches.

The technology was applied to the measurement of Hepatitis-c virus in the blood of patients directly without amplification. Complimentary DNA with the following sequence:

5″-ACC TCA CCA TAC TGC ACT CAG GCA AGC CAT TCT CTG CTG GGG GGA ATT GAT GAA TCT AGC TAC CTG GGT GGG TAA-3′ with a 20-mer poly-L-lysine at the 5′ end was custom synthesized by NBI/Genovus Inc., Plymouth, Minn. (Lot #055310).

The fluorescent probe 1-acetoxyoxypyrene-3,6,8-trisulfonyl chloride was synthesized as outlined in Dowben, R., U.S. patent application 271,161 filed Nov. 14, 1988 and European Patent Application 90900453.3 filed Nov. 14, 1988 entitled Fluorescent Immunoassays and Fluorescent Compounds and Tracers Therefor, now abandoned.

To fluorescently label the probe, 1.0 mg of 1-acetoxypyrene-3,6,8-trisulfonyl chloride was dissolved in 200 μL dry 1,3 dimethyl-3,4,5,6-tetrahydro-2(1H)-pyrimindone (DMPU; Sigma Chemical Co., St. Louis, Mo., D-7398, Lot #69F3728). 166 μg of the amino-tagged probe was dissolved in 0.5 μL DMPU. 0.25 mg of 1-acetoxypyrene-3,6,8-trisulfonyl chloride previously prepared in DMPU was added to the DNA (6:1 ratio, 10× molar excess). The reaction pH was raised to >8.0 with 10 μL of N,N-diisopropylethylamine (Sigma Chemical Co., D-3887, Lot #92H3511). The reaction was allowed to proceed for 15 hours at room temperature. The pH of the reaction mixture was subsequently raised to >10.0 with about 800 μL of 10% KOH and stirred vigorously for 30 minutes when 500 μL of H2O was added. 1 mL of 2×SSC was then added to the reaction mixture. The reaction mixture was then dialyzed against 2×SSC using Slide-A-Lyzer® dialysis cassettes (Pierce Chemical Co., Rockford, Ill.) until the fluorescence of the dialysis buffer was reduced to background levels (about 72 hours). 166 μg of conjugated DNA was then recovered in 3.5 mL SSC. 10 μL of the resulting conjugate solution gave about 65,000 fluorescence counts.

We have also labeled nucleotide sequences with 1-hydroxypyrene-3,6-disulfonic acid-8-sulfonyl-beta-alanine succinimidyl ester that has a excitation maximum at 455 nm and can be excited by pulsed blue diode lasers.

DNA was obtained from NBI/Genovus Inc. (Plymouth, Minn., Lot #44630) a ssDNA that was complementary to the probe was used as a positive control in experiments. The DNA was diluted to a concentration of 0.4 μg/μL in 0.4 M NaOH, 10 mM EDTA. 2.0 μg of the resultant denatured DNA was blotted onto ten marked areas of a Zeta Probe GT membrane (Biorad, Hercules, Calif.). Herring testes DNA was prepared in the same manner and 2.0 μg was blotted onto the Zeta Probe GT membrane to serve as a negative control. The membranes were air dried and then soaked in 0.4 M NaOH, 10 mM IEDTA for 5 minutes. The membrane was then washed three times with 2×SSC, placed into prehybridization buffer (0.5 M NaH2PO₄, 7.0% SDS, pH 7.2), and then heated at 60° C. for 10 minutes. The prehybridized membrane was then placed into 5.0 mL of prehybridization buffer containing 30 μg of pyrene-labeled cDNA and hybridized at 60° C. overnight. The hybridization solution was removed and the membrane washed three times with wash buffer (40 mM Na₂PO4, 5% SDS, pH 7.2), and then three times with distilled, deionized H2O. The membrane was then cut into squares, each containing an individual DNA sample, and the membrane squares were then placed into test tubes. 1.5 ml of 80% methanol/2% KOH was then added to each sample and the samples were vortexed to facilitate the release of the labeled probe DNA from the sample DNA-probe DNA duplexes. The releasing solution was then read in the photometer. The positive control sample gave a fluorescence intensity of approximately 14,550 counts compared with the negative control samples, which had gave a fluorescence intensity of 4,900 counts. Thus, these data demonstrate a three-fold higher fluorescence in the positive control samples versus the negative control samples. As shown herein, the present assay and the pyrene dyes used therein are highly sensitive.

The assay was performed as described above except that RNA extracted from whole blood samples obtained from patients was used. Forty blind samples were obtained from Baylor University Medical Center, Dallas, Tex. and tested for the presence of the Hepatitis-C Virus. RNA was extracted from patient serum samples using Ambion Isolation kit #1928. Samples were extracted following the Ambion protocol; 20 μL of the extract was placed on a Zeta Probe membrane strip and fixed by drying at 50° C. for one hour. The strips were blocked with a solution of 0.5 M Na2HPO4, pH 7.2, with 7% SDS. Strips were placed into test tubes and 0.5 mL of hybridizing solution containing pyrene labeled c-DNA was added to each tube and incubated for one hour at 50° C. Strips were washed 3× using 40 mM NaH2PO4, pH 7.2, 5% SDS. 0.5 mL of 20% methanol in 0.1M NaOH was added to each tube and vortexed. Strips were removed and the solution was read in the photon counting fluorimeter. Average counts for the negative control and for all negative samples was 1284.7 (range 1263-1476), while positive results averaged 14,556.9 (range 12,687-16,121) All positive results were verified by Baylor Hospital and no false positives or false negatives were observed.

What is claimed is:

1: A method for detecting a known specific target analyte using time-resolved photon counting comprising:
(a) labeling a target analyte with a fluorescent-labeled probe or probes;
(b) detecting the presence of the fluorescent-labeled probe or probes using time-resolved photon counting, where the presence of fluorescent-labeled probe or probes is indicative of the presence of the target analyte, and where a pulsed laser light is split into a plurality of beams, at least one of which illuminates the fluorescent-labeled probe or probes and at least one of which is used to measure background fluorescence.

2: The method of claim 1 in which the fluorescence-labeled probe is formed from a reaction that includes a pyrene compound.

3: The method of claim 1, where the target analyte is a nucleic acid or nucleic acids, comprising the additional steps of:
a) binding single stranded nucleic acids to a solid matrix;
b) contacting the matrix-bound nucleic acids under hybridizing conditions with the fluorescent-labeled probe or probes, the fluorescent-labeled probe or probes having portions complementary for the target analyte;
c) washing the matrix-bound nucleic acids under stringent conditions to remove the fluorescent-labeled probe or probes that are not bound or are non-specifically bound to the matrix-bound nucleic acids;
d) detecting the presence of the fluorescent-labeled probe or probes using time-resolved photon counting, where the presence of fluorescent-labeled probe or probes is indicative of the presence of the target nucleic analyte.

4: The method of claim 3, in which the nucleic acids have not been amplified by polymerase chain reaction subsequent to isolation of the nucleic acids from a biological sample.

5: The method of claim 1 in which the analyte is a nucleic acid labeled by covalent reaction of the fluorescent probe to the epsilon amino groups of a polylysine oligomer attached to the 5′ end of the nucleic acid.

6: The method of claim 1 in which the nucleic acid is labeled with multiple probes.

7: An analyte detection system using time-resolved photon counting comprising:
a) a laser light source having a pulse generator that produces exciting light pulses;
b) a beam splitter that splits the laser light source into multiple beams of light pulses;
c) a plurality of photomultiplier tubes; and
d) a multi-channel gated photon counter having at least two independent counting channels, so that at least one beam from the beam splitter passes through a first photomultiplier tube without the beam reflecting off of the fluorescent-labeled analyte, to measure the background fluorescence, and at least one beam from the beam splitter passes through a second photomultiplier tube after the beam reflects off of the fluorescent-labeled analyte to measure analyte fluorescence plus background, and where the background fluorescence is subtracted from the analyte fluorescence to detect the analyte.

8: The analyte detection system using time-resolved photon counting of claim 7, in which a mirror reflects fluorescence from the fluorescent-labeled probe or probes to the second photomultiplier tube to increase the amount of fluorescence measured.

9: The analyte detection system of claim 8 in which the mirror is a parabolic mirror.

10: The analyte detection system of claim 9 in which one or more of the photomultiplier tubes have rapid electron transit times from the cathode to first dynode.

11: The analyte detection system of claim 7 in which the photomultiplier tube is cooled.

12: The analyte detection system of claim 11 in which the photomultiplier tube is cooled to 0° C. or less.

13: The system of claim 1 in which the target analyte binding material is an antibody or antibody fragment, or other material that binds the known specific target analyte.

14: The method of claim 1 in which the target analyte is labeled with a fluorescent-labeled probe or probes by connecting the target analyte and fluorescent-labeled probe or probes with a target analyte binding material.
